(19) United States
(12) Patent Application Publication
(10) Pub. No.: US 2013/0264271 A1
(43) Pub. Date: Oct. 10, 2013

(54) BLOOD CELL SEPARATOR

(71) Applicants: SEIKO EPSON CORPORATION, Tokyo (JP); SHINSHU UNIVERSITY, Matsumoto-shi (JP)

(72) Inventors: Satomi YOSHIOKA, Shiojiri-shi (JP); Hiroshi YAGI, Tatsuno-machi (JP); Shigetaka SHIMODAIRA, Matsumoto-shi (JP)

(73) Assignees: SHINSHU UNIVERSITY, Matsumoto-shi (JP); Seiko Epson Corporation, Tokyo (JP)

(21) Appl. No.: 13/849,116

(22) Filed: Mar. 22, 2013

(30) Foreign Application Priority Data
Apr. 5, 2012 (JP) 2012-86514

(51) Int. Cl. A61M 1/36

(52) U.S. Cl. CPC A61M 1/36
USPC 210/407

(57) ABSTRACT

A blood cell separator includes a receiving container that includes an opening and a bottom surface, a tubular collection container that has a first opening, a second opening that is opposite to the first opening, and a filter that closes the second opening, and a vibration mechanism that vibrates the receiving container, at least part of the collection container being placed in the receiving container in a state in which the bottom surface of the receiving container faces the filter. The blood cell separator is thus configured so that clogging of the filter is suppressed, and the separation efficiency is improved.

Japanese Patent Application No. 2012-86514 filed on Apr. 5, 2012, is hereby incorporated by reference in its entirety.

### BACKGROUND OF THE INVENTION

The present invention relates to a blood cell separator.

A separator has been known that includes a filter for separating the desired solid phase or solid particles from a liquid-phase/solid-phase mixture, or a dispersion in which solid particles are dispersed in a liquid. Since such a filter may clog during use, a separator for which clogging of the filter is suppressed, and the separation efficiency is improved, has been desired.

As a technique for suppressing clogging of a filter, JP-A-6-269274 discloses a configuration that includes a mechanical vibration mechanism that vibrates a porous screen (filter) via a shaft. JP-A-2001-15465 discloses a configuration that includes an ultrasonic vibration mechanism that vibrates a filter by applying ultrasonic waves to a liquid using an ultrasonic device.

Clogging of a filter can be suppressed to some extent by utilizing a separator that includes the above vibration mechanism. However, a further improvement has been desired in order to more reliably suppress clogging of a filter.

### SUMMARY

The invention may provide a blood cell separator for which clogging of a filter is suppressed, and the separation efficiency is improved.

According to one aspect of the invention, there is provided a blood cell separator including:

a receiving container that includes an opening and a bottom surface;

a tubular collection container that has a first opening, a second opening that is opposite to the first opening, and a filter that closes the second opening; and

a vibration mechanism that vibrates the receiving container,

at least part of the collection container being placed in the receiving container in a state in which the bottom surface of the receiving container faces the filter.

### BRIEF DESCRIPTION OF THE SEVERAL VIEWS OF THE DRAWING

FIG. 1A is a perspective view illustrating a blood cell separator 1 according to one embodiment of the invention, and FIG. 1B is a plan view illustrating the blood cell separator 1 according to one embodiment of the invention, and a cross-sectional view taken along the line A-A in the plan view.

FIG. 2A is a cross-sectional view illustrating a first usage example of the blood cell separator 1 according to one embodiment of the invention, and FIG. 2B is a cross-sectional view illustrating a comparative example.

FIG. 3 is a table illustrating the measurement results obtained in the first usage example and the comparative example.

FIG. 4 is a graph illustrating the measurement results obtained in the second usage example.

### DETAILED DESCRIPTION OF THE EMBODIMENT

(1) According to one embodiment of the invention, there is provided a blood cell separator including:

a receiving container that includes an opening and a bottom surface;

a tubular collection container that has a first opening, a second opening that is opposite to the first opening, and a filter that closes the second opening; and

a vibration mechanism that vibrates the receiving container,

at least part of the collection container being placed in the receiving container in a state in which the bottom surface of the receiving container faces the filter.

When at least part of the collection container is placed in the receiving container in a state in which the bottom surface of the receiving container faces the filter, and the separation target liquid containing a solid component is injected through the opening of the receiving container, a filtrate that has passed through the filter moves into the collection container that is positioned above the filter with respect to the direction of gravitational force, and a residue that could not pass through the filter remains in the receiving container that is positioned under the filter with respect to the direction of gravitational force. Specifically, the flow direction with respect to the filter is opposite to the direction of gravitational force. Moreover, when the receiving container is vibrated due to the vibration mechanism, the filter is also vibrated via the receiving container and the separation target liquid. According to the above configuration, since the flow direction with respect to the filter is opposite to the direction of gravitational force, and the receiving container and the filter are vibrated due to the vibration mechanism, it is possible to suppress a situation in which the solid component contained in the separation target liquid continuously flows into the pores of the filter. This makes it possible to prevent a situation in which the filter clogs, and implement a blood cell separator that exhibits high separation efficiency. It is also possible to prevent a situation in which the solid component contained in the separation target liquid unnecessarily passes through the filter, and implement a blood cell separator that exhibits high separation efficiency. When the solid component that is contained in the separation target liquid, and has a specific gravity higher than that of the liquid contained in the separation target liquid is separated into the filtrate, it is likely that the solid component contained in the separation target liquid comes in contact with the filter as a result of stirring the separation target liquid using the vibration mechanism. This also makes it possible to implement a blood cell separator that exhibits high separation efficiency.

(2) In the blood cell separator, the receiving container and the collection container may not be secured on each other.

According to the above configuration, since the receiving container and the collection container are not secured on each other, the distance between the bottom surface of the receiving container and the filter changes with time when the receiving container is vibrated due to the vibration mechanism. Specifically, the volume of the space between the bottom surface of the receiving container and the filter easily changes with time. Therefore, since the effect of stirring the separation target liquid increases, and it is likely that a solid component contained in the separation target liquid comes in contact with the filter, a blood cell separator that exhibits high separation efficiency can be implemented.

(3) In the blood cell separator, an area of the bottom surface of the receiving container may be smaller than an area of the opening of the receiving container.

According to the above configuration, since the difference in area between the bottom surface of the receiving container and the filter is smaller than the difference in area between the opening of the receiving container and the filter, it is likely that the separation target liquid that is positioned in the vicinity of the bottom surface of the receiving container comes in contact with the filter. This makes it possible to implement a blood cell separator that exhibits high separation efficiency. Moreover, a change in height of the liquid level of the separation target liquid (i.e., the height of the interface between the separation target liquid and air) when the position of the collection container has changed relative to the receiving container can be reduced as compared with the case where the receiving container has a columnar (inner) shape. This makes it possible to suppress a situation in which the separation target liquid prevents a change in position of the collection container. Therefore, since the effect of stirring the separation target liquid increases, and it is likely that a solid component contained in the separation target liquid comes in contact with the filter, a blood cell separator that exhibits high separation efficiency can be implemented.

Exemplary embodiments of the invention are described in detail below with reference to the drawings. Note that the following exemplary embodiments do not unduly limit the scope of the invention as stated in the claims. Note also that all of the elements described below should not necessarily be taken as essential elements of the invention.

1. Configuration of Blood Cell Separator

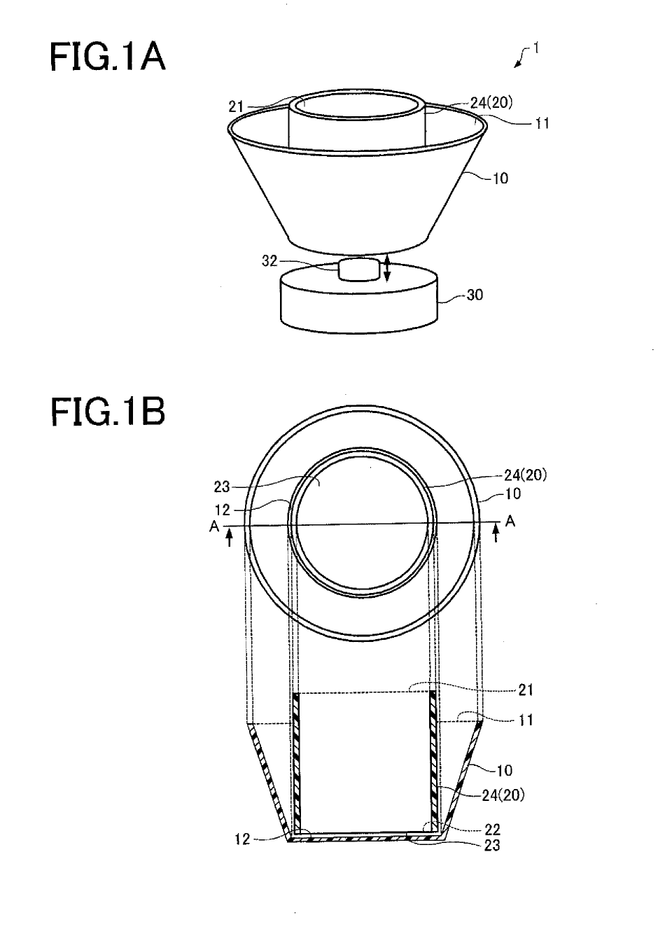

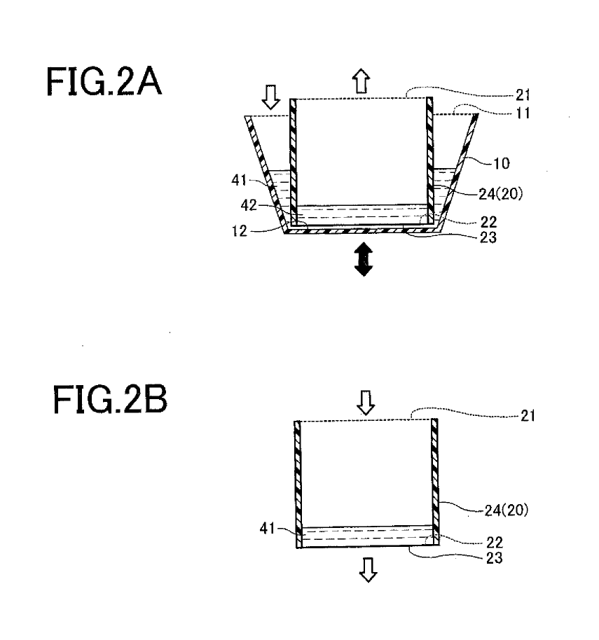

FIG. 1A is a perspective view illustrating a blood cell separator 1 according to one embodiment of the invention, and FIG. 2B is a plan view illustrating the blood cell separator 1 according to one embodiment of the invention, and a cross-sectional view taken along the line A-A.

The blood cell separator 1 according to one embodiment of the invention includes a receiving container 10 that includes an opening 11 and a bottom surface 12, a tubular collection container 20 that has a first opening 21, a second opening 22 that is opposite to the first opening 21, and a filter 23 that closes the second opening 22, and a vibration mechanism 30 that vibrates the receiving container 10, at least part of the collection container 20 being placed in the receiving container 10 in a state in which the bottom surface 12 of the receiving container 10 faces the filter 23.

The receiving container 10 has the opening 11. The opening 11 functions as an inlet for the separation target liquid. It suffices that the opening 11 have a size and a shape sufficient for placing at least part of the collection container 20 in the receiving container 10. In the example illustrated in FIGS. 1A and 1B, the opening 11 has a circular shape.

The receiving container 10 has the bottom surface 12. It suffices that the bottom surface 12 have a size and a shape sufficient for the bottom surface 12 to face the filter 23 when the collection container 20 is placed in the receiving container 10. The bottom surface 12 need not necessarily be a flat surface, but may be a surface that has partial elevations and depressions. In the example illustrated in FIGS. 1A and 113, the bottom surface 12 has a circular flat shape.

The collection container 20 has the first opening 21. The first opening 21 functions as an outlet for a filtrate that has passed through the filter 23. It suffices that the first opening 21 have a size and a shape sufficient for removing a filtrate that has passed through the filter 23. In the example illustrated in FIGS. 1A and 1B, the first opening 21 has a circular shape.

The collection container 20 has the second opening 22. It suffices that the second opening 22 have a size and a shape sufficient to provide the filter 23. In the example illustrated in FIGS. 1A and 1B, the second opening 22 has a circular shape.

The collection container 20 has the filter 23. The filter 23 is provided to close the second opening 22. In the example illustrated in FIGS. 1A and 1B, since the second opening 22 has a circular shape, the filter 23 also has a circular planar shape. Although the filter 23 has a flat surface in the example illustrated in FIGS. 1A and 1B, the filter 23 may have a curved surface. A material for forming the filter 23 may be selected from known materials (e.g., metal and resin) taking account of the composition of the separation target liquid and the like. For example, when the separation target liquid is an aqueous liquid, a hydrophilic material may be used as the material for forming the filter 23, or the surface of the filter 23 may be hydrophilized. The filter 23 has a plurality of through-holes. The pore size of the through-holes is set so that the target solid contained in the separation target liquid does not easily pass through.

The collection container 20 has the tubular body 24. The body 24 and the filter 23 may be formed integrally, or may be formed independently.

At least part of the collection container 20 is placed in the receiving container 10 in a state in which the bottom surface 12 of the receiving container 10 faces the filter 23. Specifically, when the bottom surface 12 of the receiving container 10 is positioned below the opening 11 with respect to the direction of gravitational force, at least part of the collection container 20 is placed in the receiving container 10 so that the second opening 22 and the filter 23 of the collection container 20 are positioned below the first opening 21 with respect to the direction of gravitational force. In the example illustrated in FIGS. 1A and 1B, the first opening 21 and an area around the first opening 21 are not placed in the receiving container 10. Note that the first opening 21, the second opening 22, and the filter 23 may be placed in the receiving container 10, or the entire collection container 20 may be placed in the receiving container 10.

The vibration mechanism 30 vibrates the receiving container 10. In one embodiment of the invention, the vibration mechanism 30 includes a protrusion 32. In the example illustrated in FIG. 1A, the protrusion 32 vibrates upward and downward (i.e., in the direction indicated by the two-headed arrow in FIG. 1A), and come in contact with the bottom of the receiving container 10 (i.e., the back side of the bottom surface 12) to vibrate the receiving container 10. Note that the protrusion 32 may be vibrated in an arbitrary direction. The protrusion 32 may come in contact with the bottom of the receiving container 10 at an arbitrary direction. A known vibration mechanism such as a solenoid motor or a piezoelectric device may be used as the vibration mechanism 30. In one embodiment of the invention, a solenoid motor is used as the vibration mechanism 30. The frequency of vibrations applied by the vibration mechanism 30 may be determined by experiments so that the filter 23 easily vibrates, for example.

When at least part of the collection container 20 is placed in the receiving container 10 in a state in which the bottom surface 12 of the receiving container 10 faces the filter 23, and the separation target liquid containing a solid component is injected through the opening 11 of the receiving container 10, a filtrate that has passed through the filter 23 moves into the collection container 20 that is positioned above the filter 23 with respect to the direction of gravitational force, and a residue that could not pass through the filter 23 remains in the receiving container 10 that is positioned under the filter 23 with respect to the direction of gravitational force. Specifically, the flow direction with respect to the filter 23 is opposite to the direction of gravitational force. Moreover, when the receiving container 10 is vibrated due to the vibration mechanism 30, the filter 23 is also vibrated via the receiving container 10 and the separation target liquid.

According to one embodiment of the invention, since the flow direction with respect to the filter 23 is opposite to the direction of gravitational force, and the receiving container 10 and the filter 23 are vibrated due to the vibration mechanism 30, it is possible to suppress a situation in which the solid component contained in the separation target liquid continuously flows into the pores of the filter. This makes it possible to prevent a situation in which the filter 23 clogs, and implement a blood cell separator 1 that exhibits high separation efficiency. It is also possible to prevent a situation in which the solid component contained in the separation target liquid unnecessarily passes through the filter 23, and implement a blood cell separator 1 that exhibits high separation efficiency. When the solid component that is contained in the separation target liquid, and has a specific gravity higher than that of the liquid contained in the separation target liquid is separated into the filtrate, it is likely that the solid component contained in the separation target liquid comes in contact with the filter 23 as a result of stirring the separation target liquid using the vibration mechanism 30. This also makes it possible to implement a blood cell separator 1 that exhibits high separation efficiency.

The receiving container 10 and the collection container 20 may not be secured on each other. In the example illustrated in FIGS. 1A and 1B, the receiving container 10 and the collection container 20 are not bonded or fitted to each other.

According to one embodiment of the invention, since the receiving container 10 and the collection container 20 are not secured on each other, the distance between the bottom surface 12 of the receiving container 10 and the filter 23 changes with time when the receiving container 10 is vibrated due to the vibration mechanism 30. Specifically, the volume of the space between the bottom surface 12 of the receiving container 10 and the filter 23 easily changes with time. Therefore, since the effect of stirring the separation target liquid increases, and it is likely that a solid component contained in the separation target liquid comes in contact with the filter 23, a blood cell separator 1 that exhibits high separation efficiency can be implemented.

The area of the bottom surface 12 of the receiving container 10 may be smaller than the area of the opening 11 of the receiving container 10. In the example illustrated in FIGS. 1A and 1B, the receiving container 10 is formed in the shape of a frustum so that the area of the bottom surface 12 is smaller than the area of the opening 11. In the example illustrated in FIGS. 1A and 1B, the horizontal cross-sectional area of the receiving container 10 decreases as the distance from the bottom surface 12 decreases.

According to the above configuration, since the difference in area between the bottom surface 12 of the receiving container 10 and the filter 23 is smaller than the difference in area between the opening 11 of the receiving container 10 and the filter 23, it is likely that the separation target liquid that is positioned in the vicinity of the bottom surface 12 of the receiving container 10 comes in contact with the filter 23. This makes it possible to implement a blood cell separator 1 that exhibits high separation efficiency. Moreover, a change in height of the liquid level of the separation target liquid (i.e., the height of the interface between the separation target liquid and air) when the position of the collection container 20 has changed relative to the receiving container 10 can be reduced as compared with the case where the receiving container 10 has a columnar (inner) shape. This makes it possible to suppress a situation in which the separation target liquid prevents a change in position of the collection container 20. Therefore, since the effect of stirring the separation target liquid increases, and it is likely that a solid component contained in the separation target liquid comes in contact with the filter 23, a blood cell separator 1 that exhibits high separation efficiency can be implemented.

2. Usage of Blood Cell Separator

2-1. First Usage Example

FIG. 2A is a cross-sectional view illustrating a first usage example of the blood cell separator 1 according to one embodiment of the invention, and FIG. 2B is a cross-sectional view illustrating a comparative example. Note that each white arrow in FIGS. 2A and 2B indicates the liquid flow direction, and the black two-headed arrow indicates the vibration direction due to the vibration mechanism 30. Note the same elements as those illustrated in FIGS. 1A and 1B are indicated by identical reference signs, and detailed description thereof is omitted.

In the first usage example of the blood cell separator 1 illustrated in FIG. 2A, a separation target liquid 41 was injected into the receiving container 10 through the opening 11 of the receiving container 10, and a filtrate 42 that has passed through the filter 23 was collected through the first opening 21 of the collection container 20. In the comparative example illustrated in FIG. 2B, the separation target liquid 41 was injected into the collection container 20 through the opening 21 of the collection container 20, and the filtrate 42 that has passed through the filter 23 was collected at a position under the filter 23 with respect to the direction of gravitational force.

A suspension of a human monocytic cell line THP-1 (concentration: 10⁶ cells/ml) was used as the separation target liquid 41. The filter 23 had an outer diameter of 1 cm, a thickness of 10 micrometers, and a pore size of 8 micrometers. The frequency and the amplitude of vibrations applied by the vibration mechanism 30 were 400 msec and 0.25 mm, respectively.

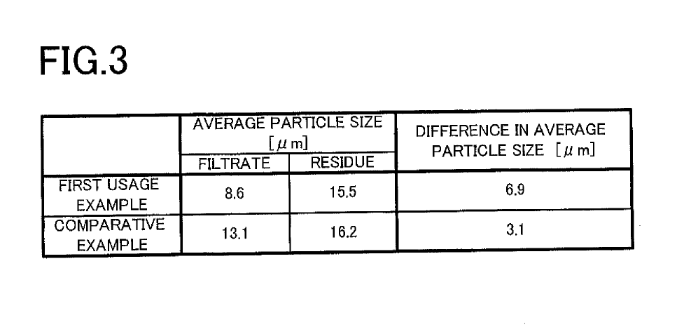

FIG. 3 is a table illustrating the measurement results obtained in the first usage example and the comparative example. The table of FIG. 3 illustrates the measurement results for the average particle size of the solid contained in the filtrate and the average particle size of the solid contained in the residue, and the difference between the average particle size of the solid contained in the filtrate and the average particle size of the solid contained in the residue in the first usage example and the comparative example. In the example of FIG. 3, the average particle size of the solid contained in the filtrate and the average particle size of the solid contained in the residue were measured using a laser diffraction particle size analyzer “SALD-300V” (manufactured by Shimadzu Corporation).

A solid such as a cell that is easily deformed tends to unnecessarily pass through the filter 23. As illustrated in FIG. 3, the difference between the average particle size of the solid contained in the filtrate and the average particle size of the solid contained in the residue was larger in the first usage example in which the blood cell separator 1 was used, as compared with the comparative example, although an identical filter was used as the filter 23. The above results suggest that a situation in which a solid unnecessarily passes through the filter 23 could be suppressed in the first usage example of the blood cell separator 1.

2-2. Second Usage Example

A second usage example of the blood cell separator 1 is described below.

Human blood was 2-fold diluted with phosphate buffered saline (PBS), and each blood component was separated by density-gradient centrifugation (1400 rpm, 30 minutes) using a reagent “Fico11”. The number of the respective blood components contained in the separation target liquid prepared by diluting the buffy coat with PBS, and the number of the respective blood components contained in a residue obtained by filtering the separation target liquid using the blood cell separator 1 were measured using a hematology analyzer (“XE-2100” manufactured by Sysmex Corporation).

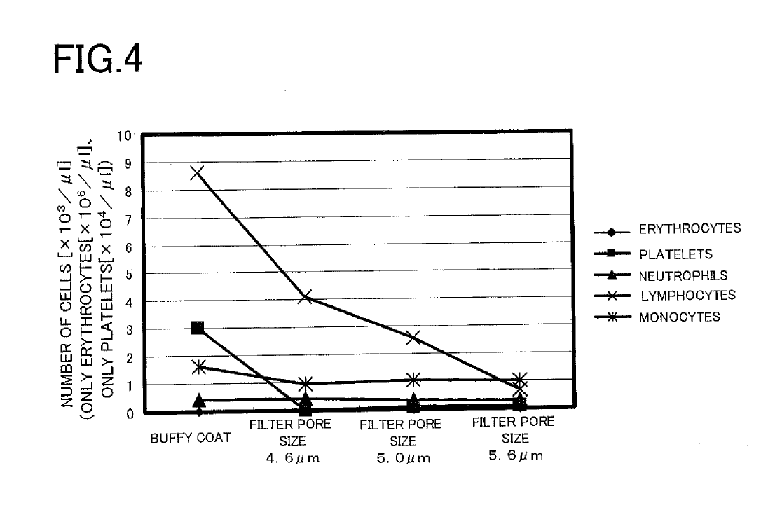

FIG. 4 is a graph illustrating the measurement results obtained in the second usage example. The graph of FIG. 4 illustrates (from left to right) the number of the respective blood components contained in the separation target liquid prepared by diluting the buffy coat with PBS, the number of the respective blood components contained in a residue obtained by filtering the separation target liquid using the filter 23 having a pore size of 4.6 micrometers, the number of the respective blood components contained in a residue obtained by filtering the separation target liquid using the filter 23 having a pore size of 5.0 micrometers, and the number of the respective blood components contained in a residue obtained by filtering the separation target liquid using the filter 23 having a pore size of 5.6 micrometers.

As illustrated in FIG. 4, a large amount of platelets and lymphocytes were present in the separation target liquid prepared by diluting the buffy coat with PBS. In contrast, the number of platelets and lymphocytes significantly decreased by filtering the separation target liquid using the blood cell separator 1. The above results suggest that platelets and lymphocytes that could not be separated by density-gradient centrifugation could be separated by the second usage example that utilized the blood cell separator 1. It was thus confirmed that the blood cell separator 1 exhibited high separation efficiency.

The results illustrated by the graph of FIG. 4 suggest that platelets can be sufficiently separated even when the pore size of the filter 23 was 4.6 micrometers, but it is necessary to increase the pore size of the filter 23 to 5.6 micrometers in order to separate lymphocytes. The above results suggest that the desired component can be separated by controlling the pore size of the filter 23.

Note that the above embodiments and the modifications thereof are merely examples, and the invention is not limited to the above embodiments and the modifications thereof. For example, a plurality of embodiments and/or a plurality of modifications may be appropriately combined.

The invention is not limited to the above embodiments and the examples. Various modifications and variations may be made of the above embodiments and the examples without departing from the scope of the invention. For example, the invention includes various other configurations that are substantially the same as the configurations described in connection with the above embodiments (e.g., a configuration having the same function, method, and results, or a configuration having the same objective and results). The invention also includes a configuration in which an unsubstantial section (element) described in connection with the above embodiments is replaced with another section (element). The invention also includes a configuration having the same effects as those of the configurations described in connection with the above embodiments, or a configuration capable of achieving the same objective as that of the configurations described in connection with the above embodiments. The invention further includes a configuration in which a known technique is added to the configurations described in connection with the above embodiments.

Although only some embodiments of the invention have been described in detail above, those skilled in the art would readily appreciate that many modifications are possible in the embodiments without materially departing from the novel teachings and advantages of the invention. Accordingly, all such modifications are intended to be included within the scope of the invention.

What is claimed is:

1. A blood cell separator comprising:
a receiving container that includes an opening and a bottom surface;
a tubular collection container that has a first opening, a second opening that is opposite to the first opening, and a filter that closes the second opening; and
a vibration mechanism that vibrates the receiving container,
at least part of the collection container being placed in the receiving container in a state in which the bottom surface of the receiving container faces the filter.

2. The blood cell separator as defined in claim 1,
the receiving container and the collection container not being secured on each other.

3. The blood cell separator as defined in claim 1,
an area of the bottom surface of the receiving container being smaller than an area of the opening of the receiving container.

4. The blood cell separator as defined in claim 1,
the filter having a flat surface.

5. The blood cell separator as defined in claim 1,
the filter having a curved surface.

6. The blood cell separator as defined in claim 1,
the filter including a hydrophilic material.

7. The blood cell separator as defined in claim 1,
the filter having a hydrophilized surface.

8. The blood cell separator as defined in claim 1,
a height from the second opening to the first opening of the collection container being greater than a height from the bottom surface to the opening of the receiving container.

9. The blood cell separator as defined in claim 1,
entirety of the collection container being placed in the receiving container.

10. The blood cell separator as defined in claim 1,
the collection container having a tubular body, and
the body and the filter being formed integrally.

11. The blood cell separator as defined in claim 1,
the collection container having a tubular body, and
the body and the filter being formed independently.
